(19) United States
(12) Patent Application Publication
(10) Pub. No.: US 2012/0088705 A1
(43) Pub. Date: Apr. 12, 2012

(54) POLYALKYLENE GLYCOL-BASED ETHER PYRROLIDONE CARBOXYLIC ACIDS, AND CONCENTRATES FOR THE PRODUCTION OF SYNTHETIC COOLING LUBRICANTS CONTAINING THE SAME

(75) Inventors: Rainer Kupfer, Hattersheim (DE); Carsten Cohrs, Frankfurt (DE); Alexander Roesch, Oppenheim (DE)

(73) Assignee: CLARIANT FINANCE (BVI) LIMITED, Tortola (VG)

(21) Appl. No.: 13/376,403

(22) PCT Filed: May 19, 2010
(86) PCT No.: PCT/EP2010/003063

§ 371 Date: Dec. 6, 2011

(30) Foreign Application Priority Data
Jun. 25, 2009 (DE) 10 2009 030 412.6

(51) Int. Cl. C10M 105/70; C07D 403/14; C07D 207/277; C07D 403/12

(52) U.S. Cl. 508/297; 548/519; 548/531

(57) ABSTRACT

The subject of the invention is an etherpyrrolidone carboxylic acid of the formula (1)

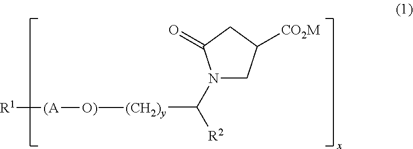

in which
R¹ is a C₁-C₆ hydrocarbon group with 1-4 oxy groups
R² is H, CH₃, CH₂CH₃
(A-O) is a (poly)alkylene glycol unit containing one or more C₂-C₄ alkoxy groups or a mixture of plural C₂-C₄ alkoxy groups, at least one alkoxy group being a butoxy or propoxy group
M is hydrogen, alkali metal, alkaline earth metal or an ammonium group
y is 1 or 2
x is 1, 2, 3 or 4.

The present invention relates to etherpyrrolidone carboxylic acids based on polyalkylene glycols and to concentrates for preparing synthetic cooling lubricant compositions that comprise these etherpyrrolidone carboxylic acids as lubricants.

A water-based concentrate for preparing synthetic cooling lubricant compositions typically comprises the following components (described by, for example, T. Mang, W. Dressel: “Lubricants and Lubrications”, Wiley-VCH, Weinheim, 2001, chapter 14, or J. P. Byers “Metalworking Fluids”, Taylor & Francis, 2006, p. 127 ff.):

| | |
|---|---|
| (1) water | 40-70% |
| (2) lubricants | 10-30% |
| (3) corrosion inhibitors | 1-20% |
| (4) pH modifiers (e.g. alkanolamines) | 1-30% |
| (5) EP/AW additives | 0-10% |
| (6) defoamers | 0-2% |
| (7) biocides | 0-5% |

(figures in percentages by weight)

The pH is situated typically in the alkaline range, generally at pH>8. Raising the pH contributes to corrosion control. In accordance with the composition above, critical significance attaches to the corrosion inhibitors and lubricants.

These synthetic cooling lubricant compositions are employed in a host of applications, particularly as grinding and cutting fluids, and are prepared at the site of use by dilution of cooling lubricant composition concentrates that already contain water.

The additives that are nowadays used already have very good properties; nevertheless, the users of cooling lubricant compositions expect continual reductions in cost, which are achievable only through the use of ever-smaller quantities of highly effective additive components.

It was an object of the present invention, therefore, to find lubricants, for use as a constituent of cooling lubricant composition concentrates, that produce lubricity effects comparable with those of the prior art at significantly lower use concentrations.

Lubricants used very frequently in water-soluble cooling lubricant composition concentrates are polyalkylene glycols (T. Mang, W. Dressel: “Lubricants and Lubrications”, Wiley-VCH, Weinheim, 2001, p. 378), which are obtainable by sequential or random polymerization of alkylene oxides, preferably of ethylene oxide and propylene oxide. The mode of action of these lubricants is based on “hydrodynamic lubrication”. The polyalkylene glycols have a cloud point—that is, at a certain temperature, the polymers become insoluble in water. If this temperature is reached as a result of the heat given off during grinding or cutting of metal, the polymers come out of the solution and form a thin lubricating film between metal and tool. This property, however, also produces a disadvantage: the polyglycols leave behind tacky residues, particularly when relatively old grinding and cutting fluids, after long use, have a high lime soap fraction and electrolyte content, which lowers the cloud point.

WO-00/53701 describes polyalkylene glycols terminated by carboxyl groups as lubricants. The carboxyl groups are introduced either by reacting a polyalkylene glycol with anhydrides in a simple condensation reaction or else by forming an ether carboxylic acid by reaction of the polyalkylene glycols with monochloroacetic salt. Following neutralization by the alkaline modification of the pH, these lubricants have no cloud points, or have very high cloud points, but still attain lubrication values comparable with those of the initial polyalkylene glycols. A reduced oxygen demand on disposal is quoted as an advantage.

As a result, however, of the hydrolysis that occurs under the alkaline conditions, the esters prepared by reaction with the anhydrides are very limited in their lifetime, and so very soon only the polyalkylene glycols are present free again. A disadvantage of the ether carboxylic acids is the waste product, obtained in the preparation process, of at least 4 mol of sodium chloride per mole of difunctional polyalkylene glycol, and the low space-time yield in the preparation process, leading overall to high costs. A further problem is the attainment of a complete conversion. If the polyalkylene glycol itself is not water-soluble, e.g., a polypropylene glycol, residues of the free glycol lead to unwanted clouding or biphasicity of the concentrate.

Etherpyrrolidone carboxylic acids (also “EPC” hereinafter) and their preparation are known in the prior art. Accordingly, U.S. Pat. No. 4,304,690, U.S. Pat. No. 4,298,708 and U.S. Pat. No. 4,235,811 describe by way of example the preparation of etherpyrrolidone carboxylic acids on the basis of fatty alcohol (poly)ether amines and the use thereof in laundry detergents and as a catalyst for producing polyurethane foams. These pyrrolidone carboxylic acids, originating from fatty alcohols and their alkoxylates, are oil-soluble and water-dispersible and, following neutralization, can be used in emulsifiable cooling lubricant composition concentrates as an emulsifier and corrosion inhibitor.

GB-A-1 323 061 claims hydraulic fluids which comprise pyrrolidone carboxylic acids, it being possible for said acids inter alia to be polyalkylene glycol-based etherpyrrolidonemonocarboxylic and -dicarboxylic acids. GB-A-1 323 061, however, describes only etherpyrrolidone carboxylic acids originating from (poly)ethylene glycol amines, namely only the short-chain polyamine H163 (9-hydroxy-4,7-dioxanonanamine) and also its monoethanolamine derivative. These EPC are used as corrosion inhibitors in hydraulic fluids. A further application described is their use as a corrosion inhibitor in acidic cleaning products. No lubricating effect is taught.

It has now been found that etherpyrrolidone carboxylic acids of the formula 1 which as well as ethylene glycol units also comprise propylene glycol units, or, more particularly, comprise only propylene glycol units, represent particularly effective lubricants in alkaline cooling lubricant compositions. The effect is particularly pronounced when the etherpyrrolidone carboxylic acids contain long polyalkylene glycol units, more particularly polypropylene glycol units—in other words, have a high molar weight. These etherpyrrolidone carboxylic acids are able to replace polyalkylene glycols and other prior-art lubricants in concentrates for the preparation of synthetic (water-soluble) cooling lubricant compositions, and significantly lower use concentrations need be used. The preparation of these etherpyrrolidone carboxylic acids can be accomplished, in a manner analogous to the fatty alcohol etherpyrrolidone carboxylic acids, by the reaction of itaconic acids with the polyetheramines. These polyetheramines are in some cases available commercially under the trade name Polyetheramine (BASF) or Jeffamine® (Huntsman), or can be obtained either by aminolysis of suitable polyalkylene glycols with ammonia or by reaction of polyalkylene glycols with acrylonitrile, with subsequent hydrogenation of the nitrile.

The invention accordingly provides etherpyrrolidone carboxylic acids of the formula (1)

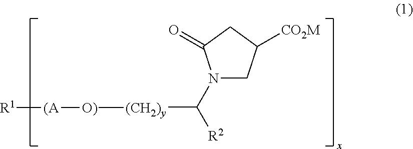

in which
R¹ is a C₁-C₆ hydrocarbon group with 1-4 oxy groups
R² is H, CH₃, CH₂CH₃
(A-O) is a (poly)alkylene glycol unit containing one or more C₂-C₄ alkoxy groups or a mixture of plural C₂-C₄ alkoxy groups, at least one alkoxy group being a butoxy or propoxy group
M is hydrogen, alkali metal, alkaline earth metal or an ammonium group
y is 1 or 2
x is 1, 2, 3 or 4.

The invention further provides concentrates for preparing synthetic cooling lubricant compositions which comprise etherpyrrolidone carboxylic acids of the formula (1), and the use of these concentrates for preparing synthetic cooling lubricant compositions.

The polyalkylene glycol unit (A-O) in formula (1) comprises one or a sequence of two or more C₂-C₄ alkylene oxide units, which may be different from one another. The different units may be arranged in random order or else sequentially (blockwise). Where (A-O) is a single alkoxy group, (A-O) denotes a propoxy or butoxy group. Where (A-O) stands for more than one alkoxy group, (A-O) comprises at least one propoxy group or at least one butoxy group. These polyalkylene glycol units are formed in the first step of the preparation of the etherpyrrolidone carboxylic acids through alkoxylation of a hydroxyl group of a starter alcohol with one or more C₂-C₄ alkylene oxides to form a polyalkylene glycol, and the alkoxylation with two or more alkylene oxides may take place either with a mixture of the alkylene oxides or else sequentially. In accordance with the invention, however, one of the alkylene oxides must be other than ethylene oxide.

In one preferred embodiment, the polyalkylene glycol units (A-O) comprise sequentially arranged alkoxy groups. In another preferred embodiment, the fraction of propoxy and/or butoxy groups is more than 20 mol %, more particularly more than 50 mol %. In one particularly preferred embodiment, there are purely polypropylene glycol units.

The group -(A-O)— in one preferred embodiment stands for a poly(oxyalkylene) group of the formula (2)

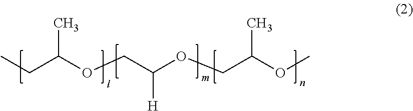

in which

l is, a number from 0 to 200,

m is a number from 0 to 200,

n is a number from 0 to 200,

and l+n is at least 1.

l is preferably a number from 1 to 10.

m is preferably a number from 20 to 50.

n is preferably a number from 1 to 10.

In a further preferred embodiment, l is zero, m is a number from 1 to 50, more particularly 5 to 25, and n is 1 to 200, more particularly 2 to 25.

In a further preferred embodiment, l and m are zero, and n is 1 to 50, more particularly 2 to 30.

The number of alkoxy groups present in the (A-O) units of the etherpyrrolidone carboxylic acids of the invention is best defined through the molecular weight of the polyalkylene glycol groups. Hence the number-average molecular weight of the polyalkylene glycol group is generally at least 75 g/mol, preferably greater than 200 g/mol, more preferably 1000-10 000 g/mol. The number-average molecular weight of the polyalkylene glycol group may be determined, for example, by gel permeation chromatography (GPC) on the basis of the unit R¹-[(A-O)—H]ₓ. The activity of the etherpyrrolidone carboxylic acids of the invention increases in line with the molecular weight of the polyalkylene glycols used as intermediates. The molecular weight, however, is limited by the incipient insolubility particularly of those high molecular weight etherpyrrolidone carboxylic acids that contain a high fraction of propoxy or butoxy groups, at a pH>7.

R¹ is a radical obtained by the formal abstraction of the hydroxyl hydrogen atom of a mono-, di-, tri- or tetrahydric alcohol which comprises 1 to 7 carbon atoms. This alcohol preferably comprises 2 to 6 carbon atoms. Moreover, R¹ is preferably an aliphatic group. Formal abstraction in the present context means that the hydroxyl hydrogen atom is omitted. Starting from methanol, CH₃OH, therefore, R¹ is CH₃O; starting from ethylene glycol, HO—CH₂—CH₂—OH, R¹ is either O—CH₂—CH₂—OH or O—CH₂—CH₂—O. The individual oxygen atom —O is referred to here as oxy group. The number of oxy groups corresponds to x. The number of oxy groups in the case of di, tri- or tetrahydric alcohols may be less than or equal to the number of OH groups of the alcohol. Preferred alcohols are monoalcohols of the formula R³—OH with R³=C₁-C₄, i.e., methanol, ethanol, n-propanol, n-butanol, isopropanol, isobutanol, and tert-butanol. Also suitable are diols of the formula OH—R⁴—OH, with R⁴=C₂-C₆ alkylene, examples being ethylene glycol, 1,2- and 1,3-propylene glycol, butanediol, hexanediol, neopentylglycol, triols such as glycerol or trimethylolpropane, and tetraols such as pentaerythritol.

In one preferred embodiment, the group R¹=CH₃O—; in a further-preferred embodiment, it is a difunctional group, particularly ethylene glycol and 1,2-propylene glycol.

In industrial production, it is not absolutely necessary to start from the starter unit R¹H; the starter alcohol may already comprise one or more alkoxy groups, which are then alkoxylated further. Suitable starter alcohols, then, are therefore methyl glycol, methyl diglycol, methyl triglycol, butyl glycol, butyl diglycol, butyl triglycol, diethylene glycol, triethylene glycol, tetraethylene glycol, low molecular weight polyalkylene glycols, dipropylene glycol, tripropylene glycol, butylpropylene glycol, butyldipropylene glycol, and low molecular weight polypropylene glycols.

y and R² are defined by the nature of the reaction of the polyalkylene glycols to form the polyalkylene glycol etheramines. If the etheramine has come about through aminolysis of a polyalkylene glycol, then y is 1 and R² is a product of the end group of the polyalkylene glycol, and hence may be hydrogen or a methyl or ethyl group. If the etheramine has been prepared by addition reaction of acrylonitrile and subsequent hydrogenation, then y is 2 and R² is hydrogen. In one preferred embodiment, y is 1 and R² is CH₃. In order to avoid problems in the application of the completed etherpyrrolidone carboxylic acids (a poor water solubility at pH>7), a high degree of conversion is preferred in the reaction of the polyalkylene glycols to form the polyalkylene glycol etheramines, this degree of conversion being at least 95%, preferably 97%, more preferably >99%.

A series of etheramines having different alkoxy groups, molecular weights, and functionalities are available commercially, for example, under the name Jeffamine® or Polyetheramine.

The number x can be 1 to 4 and indicates the number of the amino group. If the etheramine carries one amino group, x is 1; if it carries two, x is 2, and so on.

The reaction of the amine functions with itaconic acid produces the pyrrolidone carboxylic acid units. By using a deficit amount of itaconic acid it is possible to obtain lower carboxylic acid contents, but to some extent there is formation of polyamides. The byproducts, moreover, are less soluble, and so a high degree of conversion is preferred. It is particularly preferred to aim for a degree of conversion >99%. Of likewise great importance is an efficient ring closure to form the pyrrolidone, since the secondary amine intermediate in the end application gives rise to regulatory problems in certain countries, especially Germany. It is therefore preferred to observe a low amine content of <1% by weight, and with particular preference the target residual amine content is <0.5% by weight.

Deserving of emphasis as a particular advantage of the process is the fact that each reaction step is an addition step or condensation step, which means that all of the raw materials remain in the product (with the exception of water), and so no wastes at all are produced.

In a further preferred form, all functional groups of the original alcohol are converted into pyrrolidone carboxylic acid groups, and so x reflects the number of functional groups of the alcohol.

The etherpyrrolidone carboxylic acids obtained in this way can be used for producing cooling lubricant composition concentrates which prior to use are diluted with water to form the ready-to-use cooling lubricant compositions. Typical dilution ratios in this context are 1:1-1:100, preferably between 1:25 and 1:10 parts of concentrate to water. As compared with the lubricants known from the prior art, smaller quantities are needed here in order to achieve comparable lubricity effects, as measured, for example, on a Reichert friction-wear balance. Since the etherpyrrolidone carboxylic acids of the invention do not exhibit a cloud point at the typical pH levels of a synthetic metalworking fluid, there are no disruptive residues at all left on the workpieces or tools, and the fluids have a long lifetime on account of their high stability with respect to lime soaps and electrolytes. The concentration in the concentrate can be 0.1-60% by weight, preferably 1-25% by weight, more preferably 5-15% by weight, and is selected such that a sufficient lubricity effect is obtained in the application, as determinable, for example, on the Reichert friction-wear balance. The etherpyrrolidone carboxylic acids themselves need not be soluble in distilled water; it is sufficient, or even preferred, for them to be soluble, through the use of neutralizing agent, at a pH>7.

Besides water as solvent, these concentrates comprise the etherpyrrolidone carboxylic acids of the invention as lubricants and also a neutralizing agent for adjusting the pH, which following dilution to the final working concentration is between 7-11, preferably 8-10, more preferably between 8.5-9.5.

Neutralizing agents suitable for this purpose are amines of the formula (3)

NR⁵R⁶R⁷ (3)

in which

R⁵, R⁶ and R⁷ independently of one another are hydrogen or a hydrocarbon radical having 1 to 100 carbon atoms.

In a first preferred embodiment, R⁵ and/or R⁶ and/or R⁷ independently of one another are an aliphatic radical. This radical has preferably 1 to 24, more preferably 2 to 18, and especially 3 to 6 carbon atoms. The aliphatic radical may be linear, branched or cyclic. It may also be saturated or unsaturated. The aliphatic radical is preferably saturated. The aliphatic radical may carry substituents such as, for example, hydroxyl, C₁-C₅-alkoxy, cyano, nitrile, nitro and/or C₅-C₂₀ aryl groups such as, for example, phenyl radicals. The C₅-C₂₀ aryl radicals may in turn be substituted optionally by halogen atoms, halogenated alkyl radicals, C₁-C₂₀ alkyl, C₂-C₂₀ alkenyl, hydroxyl, C₁-C₅ alkoxy such as, for example, methoxy, amide, cyano, nitrile and/or nitro groups. In one particularly preferred embodiment, R⁵ and/or R⁶ and/or R⁷ independently of one another are hydrogen or a C₁-C₆ alkyl, C₂-C₆ alkenyl or C₃-C₆ cycloalkyl radical and especially an alkyl radical having 1, 2 or 3 carbon atoms. These radicals may carry up to three substituents. Particularly preferred aliphatic radicals R⁵ and/or R⁶ and/or R⁷ are hydrogen, methyl, ethyl, hydroxyethyl, n-propyl, isopropyl, hydroxypropyl, n-butyl, isobutyl and tert-butyl, hydroxybutyl, n-hexyl, cyclohexyl, n-octyl, n-decyl, n-dodecyl, tridecyl, isotridecyl, tetradecyl, hexadecyl, octadecyl, and methylphenyl.

In a further preferred embodiment, R⁵ and R⁶, together with the nitrogen atom to which they are attached, form a ring. This ring has preferably 4 or more such as, for example, 4, 5, 6 or more ring members. Preferred further ring members in this case are carbon, nitrogen, oxygen, and sulfur atoms. The rings may in turn carry substituents such as, for example, alkyl radicals. Suitable ring structures are, for example, morpholinyl, pyrrolidinyl, piperidinyl, imidazolyl, and azepanyl radicals. In one preferred embodiment, R⁷ then is H or an alkyl radical having 1 to 12 carbon atoms.

In a further preferred embodiment, R⁵, R⁶ and/or R⁷ independently of one another are an optionally substituted C₆-C₁₂ aryl group or an optionally substituted heteroaromatic group having 5 to 12 ring members.

In another preferred embodiment, R⁵, R⁶ and/or R⁷ independently of one another are an alkyl radical which is interrupted by heteroatoms. Particularly preferred heteroatoms are oxygen and nitrogen.

Hence R⁵, R⁶ and/or R⁷ independently of one another are preferably radicals of the formula (4)

—(R⁸—O)ₐ—R⁹ (4)

in which
R⁸ is an alkylene group having 2 to 6 carbon atoms and preferably having 2 to 4 carbon atoms such as, for example, ethylene, propylene, butylene or mixtures thereof,
R⁹ is hydrogen, a hydrocarbon radical having 1 to 24 carbon atoms or a group of the formula —R⁸—NR¹⁰R¹¹
a is a number between 2 and 50, preferably between 3 and 25, and more particularly between 4 and 10, and
R¹⁰, R¹¹ independently of one another are hydrogen, an aliphatic radical having 1 to 24 carbon atoms and preferably 2 to 18 carbon atoms, an aryl group or heteroaryl group having 5 to 12 ring members, a poly(oxyalkylene) group having 1 to 50 poly(oxyalkylene) units, the polyoxyalkylene units deriving from alkylene oxide units having 2 to 6 carbon atoms, or R¹⁰ and R¹¹, together with the nitrogen atom to which they are attached, form a ring having 4, 5, 6 or more ring members.

With further preference, R⁵, R⁶ and/or R⁷ independently of one another are radicals of the formula (5)

—[R¹²—N(R¹³)]_{b}—(R¹³) (5)

in which
R¹² is an alkylene group having 2 to 6 carbon atoms and preferably having 2 to 4 carbon atoms such as, for example, ethylene, propylene or mixtures thereof,
each R¹³ independently of any other is hydrogen, an alkyl or hydroxyalkyl radical having up to 24 carbon atoms such as, for example, 2 to 20 carbon atoms, a polyoxyalkylene radical —(R⁸—O)ₚ—R⁹, or a polyiminoalkylene radical —[R¹²—N(R¹³)]_{q}—(R¹³), where R⁸, R⁹, R¹², and R¹³ have the definitions given above and q and p independently of one another are 1 to 50, and
b is a number from 1 to 20 and preferably 2 to 10 such as, for example, three, four, five or six.

The radicals of the formula (5) contain preferably 1 to 50, more particularly 2 to 20, nitrogen atoms.

Particularly preferred neutralizing agents are water-soluble alkylamines such as methylamine, dimethylamine, trimethylamine, ethylamine, diethylamine, triethylamine, propylamine and longer-chain mono-, di-, and trialkylamines, provided they have a water solubility of at least 1% by weight, preferably 1%-5% by weight. The alkyl chains in these cases may be branched. Also suitable are oligoamines such as ethylenediamine, diethylenetriamine, triethylenetetramine, tetraethylenepentamine, their higher homologs, and mixtures of these. Other suitable amines in this series are the alkylated, especially methylated, representatives of these oligoamines, such as N,N-dimethyldiethyleneamine, N,N-dimethylpropylamine, and longer-chain and/or more highly alkylated amines of the same structure principle. Particularly suitable in accordance with the invention are alkanolamines such as monoethanolamine, diethanolamine, triethanolamine, diglycolamine, triglycolamine and higher homologs, methyldiethanolamine, ethyldiethanolamine, propyldiethanolamine, butyldiethanolamine, and longer-chain alkyldiethanolamines, it being possible for the alkyl radical to be cyclic and/or branched. Further suitable alkanolamines are dialkylethanolamines such as dimethylethanolamine, diethylethanolamine, dipropylethanolamine, dibutylethanolamine, and longer-chain dialkylethanolamines, it also being possible for the alkyl radical to be branched or cyclic. Further in the sense of the invention it is also possible to use aminopropanol, aminobutanol, aminopentanol, and higher homologs, and also the corresponding mono- and dimethylpropanolamines and longer-chain mono- and dialkylamino alcohols. Suitable not least are specific amines such as 2-amino-2-methylpropanol (AMP), 2-aminopropanediol, 2-amino-2-ethylpropanediol, 2-aminobutanediol and other 2-aminoalkanols, aminoalkylamine alcohols, tris(hydroxymethyl)aminomethane, and also endcapped representatives such as methylglycolamine, methyldiglycolamine and higher homologs, di(methylglycol)amine, di(methyldiglycol)amine and the higher homologs of these, and also the corresponding triamines and polyalkyene glycol amines (e.g., Jeffamine®). Used typically and in the sense of the invention are mixtures of the abovementioned amines for the purpose of setting desired pH levels. The concentration of these amines in the concentrate can be 1% -60% by weight, preferably 10% -30% by weight, more preferably 15% -25% by weight, and is selected such that the desired pH is obtained following dilution.

The effect of nitrogen-containing neutralizing agents is that M is an ammonium group. The concept of the ammonium group in relation to M means that this group may also be substituted. The nature of the substituents arise from the nature of the nitrogen-containing neutralizing agents, as described above.

Other suitable neutralizing agents are the oxides and hydroxides of the alkali metals and/or alkaline earth metals, such as lithium hydroxide, sodium hydroxide, potassium hydroxide, calcium hydroxide, and calcium oxide, for example.

If, however, a cloud point is desired, in order to obtain cloud point defoaming, for example, then a polyalkylene glycol can be added, but is used in significantly smaller quantities than in the case of use as a lubricant. Accordingly, the concentrate may contain at most 10% by weight, preferably 0.1% -5% by weight, more preferably 0.5% -1.5% by weight, of polyalkylene glycol.

Suitable polyalkylene glycols have the same fundamental structure as the polyalkylene glycols suitable as raw material for preparing the etherpyrrolidone carboxylic acids, but the proportion of the various alkoxy groups is selected such that the desired cloud point is attained. In one preferred embodiment, the compounds in question are EO-PO-EO block polymers which themselves have a cloud point (1% in water) of 25-90° C., preferably 30-80° C., more particularly 30- 60° C.

In accordance with the invention, the cooling lubricant composition concentrates further comprise water-soluble corrosion inhibitors in amounts of 0.1% -60% by weight, preferably 5% -25% by weight, more preferably 10% -20% by weight.

Suitable corrosion inhibitors are benzenesulfonamidocaproic acid, toluenesulfonamidocaproic acid, N-methylbenzenesulfonamidocaproic acid, N-methyltoluenesulfonamidocaproic acid (all formula (6)), alkanoylamidocarboxylic acids, particularly isononanoylamidocaproic acid (formula (7)), and triazine-2,4,6-tris(aminohexanoic acid) (formula (8)), and the alkali metal, alkaline earth metal, and amine salts of the compounds of the formulae (6)-(8).

a) toluene- or benzenesulfonamidocaproic acids (formula (6))

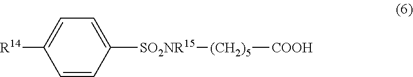

with R¹⁴, R¹⁵=H or CH₃

b) isononanoylamidocaproic acid (formula (7))

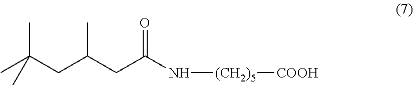

c) triazine-trisaminohexanoic acid (formula (8))

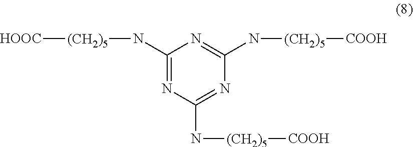

Other known and suitable corrosion inhibitors are linear or branched C₆ to C₈ carboxylic acids such as, for example, octanoic acid, 2-ethylhexanoic acid, n-nonanoic acid, n-decanoic acid, n-isodecanoic acid, dicarboxylic acids such as succinic acid, adipic acid, maleic acid, citric acid, and also longer-chain dicarboxylic acids such as decanedioic acid, undecanedioic acid or dodecanedioic acid, it being possible for the chains to be branched or else cyclic, and polycarboxylic acids. Further suitable corrosion inhibitors are alkanesulfonamides, alkanesulfonamidocarboxylic acids, and phthalic monoamides. It is also possible, furthermore, to use salts of the compounds set out above.

One widespread inorganic corrosion inhibitor is boric acid, and the salts, amides, and esters thereof.

Where the salts of one of the abovementioned corrosion inhibitors are used, the salts in question are preferably salts formed by reaction of the free acids with a neutralizing agent present in the hydraulic fluid.

Certain applications necessitate the use of EP/AW additives for reducing the abrasion. As EP/AW additives, in accordance with the invention, the concentrates may receive, for example, water-soluble phosphoric esters, their salts, and also water-insoluble phosphoric esters, which are converted into their salts by the neutralizing agents and become soluble. Suitable phosphoric esters are the monoesters and diesters of ethanol, butanol, propanol, methanol, hexanol, octanol, isooctanol, phenol, and further higher alcohols, ethylene glycol, propylene glycol, butyl glycol, butyl diglycol, and mixtures thereof, and also the monoesters and diesters of ethoxylated alcohols, and mixtures thereof.

Further suitable EP/AW additives are water-soluble sulfur compounds such as, for example, sulfides, disulfides, mercaptobenzothiazole, sulfurized acrylic acid, dithiobis(thiazoles), e.g., dimercaptothiodiazole and its salts, dithiodicarboxylic acids, especially dithio(diarylcarboxylic acids) such as, for example, dithiodibenzoic acid and its salts.

The concentration of the EP/AW additives is generally between 0.1% and 20% by weight, preferably between 1% and 10% by weight.

In addition to the additives outlined, the concentrates may in accordance with the invention also comprise biocides, defoamers, water softeners or solubilizers, e.g., butyl glycol or butyl diglycol.

The cooling lubricant composition concentrates can be produced by simple mixing of the additives in any order. In order to avoid highly viscous phases, however, it is advisable first to introduce the water and then to add the immediately water-soluble additives in any order. After that, the neutralizing agents can be added, followed by, the additives which become water-soluble only through neutralization. The etherpyrrolidone carboxylic acids can be added at any desired point. In one preferred embodiment, the etherpyrrolidone carboxylic acids are not soluble in pure water, but only at pH levels >7. In these cases it is preferred first to dissolve the neutralizing agents in water and then to add the etherpyrrolidone carboxylic acids.

For use as a cooling lubricant composition, the concentrates thus produced are diluted with distilled water or else other service water or mains water in a ratio of 1:1 to 1:100, generally between 1:25 and 1:10 parts of concentrate to water, and used.

### EXAMPLES

### Example 1-11

### General Protocol for Preparing N-Substituted 5-oxopyrrolidine-3-carboxylic acids

In a standard stirring apparatus, 1 equivalent of amine is introduced and heated to 50° C. with stirring. Then, in portions, 1 equivalent of itaconic acid is added and the reaction mixture is heated slowly to 180° C. During the progress of the reaction, 1 equivalent of water of reaction is removed by distillation. The product obtained is characterized by means of acid number (AN) and basic nitrogen (bas.-N).

### Example 1: (Comparative)

133 g of 3-(2-methoxyethoxy)propylamine and 130 g of itaconic acid gave 240 g of 5-oxo-[3-(2-methoxyethoxy)propyl]pyrrolidine-3-carboxylic acid with AN=226.5 mg KOH/g and bas.-N<0.1%.

### Example 2: (Comparative)

103 g of 4,7,10-trioxatridecane-1,13-diamine and 65 g of itaconic acid gave 155 g of 5-oxo[4,7,10-trioxatridecane-1,13-di]pyrrolidine-3-carboxylic acid with AN=271.4 mg KOH/g and bas.-N<0.1%.

### Example 3: (Comparative)

105 g of 2-(2-aminoethoxy)ethanol and 130 g of itaconic acid gave 210 g of 5-oxo-[2-hydroxyethylethoxy]pyrrolidine-3-carboxylic acid with AN=174.3 mg KOH/g and bas.-N<0.1%.

### Example 4

230 g (1 mol) of Polyetheramin® D-230 (polypropylene glycol diamine having an average molecular weight of 230 g/mol) and 260 g (2 mol) of itaconic acid gave 454 g of product with bas.-N<0.1% and AN of 251 mg KOH/g as pale yellow liquid.

### Example 5

442 g (1 mol) of Polyetheramin® D-400 (polypropylene glycol diamine having an average molecular weight of 400 g/mol) and 260 g (2 mol) of itaconic acid gave 665 g of product with bas.-N<0.1% and AN of 164 mg KOH/g as pale yellow liquid.

### Example 6

500 g (0.25 mol) of Polyetheramin® D-2000 (polypropylene glycol diamine having an average molecular weight of 2000 g/mol) and 65 g (0.5 mol) of itaconic acid gave 546 g of product with bas.-N<0.1% and AN of 52 mg KOH/g as pale yellow liquid.

### Example 7

514 g (0.25 mol) of Jeffamine® M-2070 (methylpoly(ethoxy)poly(propoxy)monoamine having an average molecular weight of about 2000 g/mol) and 32.5 g (0.25 mol) of itaconic acid gave 535 g of product with bas.-N<0.1% and AN of 25.1 mg KOH/g as yellow liquid.

### Example 8

611 g (1 mol) of Jeffamin® ED-600 (PO-EO-PO block polymer-based diamine having an average molecular weight of 600 g/mol) and 260 g (2 mol) of itaconic acid gave 835 g of product with bas.-N<0.1% and AN of 130 mg KOH/g as pale yellow liquid.

### Example 9

518 g (0.25 mol) of Jeffamine® ED-2003 (PO-EO-PO block polymer-based diamine having an average molecular weight of 2000 g/mol) and 65 g (0.5 mol) of itaconic acid gave 573 g of product with bas.-N<0.1% and AN of 47 mg KOH/g as yellow liquid.

### Example 10

98 g (0.2 mol) of Polyetheramin® T 403 (trimethylolpropanetris(polypropylene glycol amine) having an average molecular weight of 440 g/mol) and 78 g (0.6 mol) of itaconic acid gave 160 g of product with bas.-N<0.1% and AN of 198 mg KOH/g as'yellow liquid.

### Example 11

226 g (0.04 mol) of Polyetheramin® T 5000 (glyceryltris(polypropylene glycol amine) having an average molecular weight of 5000 g/mol) and 15.6 g (0.12 mol) of itaconic acid gave 230 g of product with bas.-N<0.1% and AN of 27 mg KOH/g as yellow liquid.

The etherpyrrolidone carboxylic acids from examples 1-11 were reacted with a double excess of triethanolamine to give the actual water-soluble lubricant, and, in aqueous solution, measurements were made of the behavior toward hard water, the cloud point, and the lubricity on the Reichert friction-wear balance (loading: 1.5 kg/travel 100 m/steel rollers/results of the wear mark in mm²). As a comparative, measurements were carried out on the polyalkylene glycols Genapol® PN30 and Genapol® B from the prior art. Examples 1-3 correspond to the prior art from GB-A-1 323 061, i.e., etherpyrrolidone carboxylic acids originating from (poly)ethylene glycol amines. For ease of legibility, the term “etherpyrrolidone carboxylic acid” has been abbreviated in the table as EPC. In cases where the wear mark was >30 mm², no lower concentration was measured.

TABLE 1
Properties and lubricity values of the etherpyrrolidone carboxylic acids

| | Gena- | Gena- | | | | | | | | | | | |
|---|---|---|---|---|---|---|---|---|---|---|---|---|---|
| Starting | pol ® | pol ® | Example 1 | Example 2 | Example 3 | Exam- | Exam- | Exam- | Exam- | Exam- | Exam- | Exam- | Exam- |
| substance | PN 30 | B | (C) | (C) | (C) | ple 4 | ple 5 | ple 6 | ple 7 | ple 8 | ple 9 | ple 10 | ple 11 |
| Composition | 100% | 100% | EPC: | EPC: | EPC: | EPC: | EPC: | EPC: | EPC: | EPC: | EPC: | EPC: | EPC: |
| of the lubricant | Gena- | Gena- | 47% | 25% | 25% | 45% | 53% | 75% | 88% | 47% | 75% | 68% | 93% |
| | pol ® | pol ® | TEA: | TEA: | TEA: | TEA: | TEA: | TEA: | TEA: | TEA: | TEA: | TEA: | TEA: |
| | PN30 | B | 53% | 75% | 75% | 55% | 47% | 25% | 12% | 53% | 25% | 32% | 7% |
| Appearance | clear | clear | clear | clear | clear | clear | clear | clear | clear | clear | clear | clear | clear |
| (20° C.) in | | | | | | | | | | | | | |
| water of | | | | | | | | | | | | | |
| 20° dH | | | | | | | | | | | | | |
| [German | | | | | | | | | | | | | |
| hardness] | | | | | | | | | | | | | |
| Cloud point | about | about | >95° C. | >95° C. | >95° C. | >95° C. | >95° C. | >95° C. | >95° C. | >95° C. | >95° C. | >95° C. | >95° C. |
| 1% in water | 30° C. | 56° C. | | | | | | | | | | | |
| pH, 1% in | 7 | 7 | 8.6 | 8.6 | 8.6 | 8.1 | 8.0 | 8.1 | 8.1 | 8.3 | 8.3 | 8.2 | 8.1 |
| water | | | | | | | | | | | | | |
| Reichart¹⁾ | | | | | | | | | | | | | |
| result conc. | | | | | | | | | | | | | |
| in H₂O | | | | | | | | | | | | | |
| 10% | 12 mm² | 14 mm² | 31 mm² | 21 mm² | 28 mm² | 19 mm² | 16 mm² | 12 mm² | 10 mm² | 12 mm² | 12 mm² | 11 mm² | 5 mm² |
| 5% | 12 mm² | 16 mm² | | 33 mm² | 31 mm² | 24 mm² | 22 mm² | 12 mm² | 13 mm² | 14 mm² | 12 mm² | 16 mm² | 6 mm² |
| 2.5% | | | | | | | 28 mm² | 13 mm² | 14 mm² | 18 mm² | 14 mm² | 22 mm² | 10 mm² |
| 0.5% | 24 mm² | 22 mm² | | | | 37 mm² | 34 mm² | 13 mm² | 33 mm² | 33 mm² | 18 mm² | 32 mm² | 18 mm² |

¹⁾loading 1.5 kg, steel rollers, 100 m travel, rotary speed 1.6 m/s; blank value for water: 38 mm²

The polyalkylene glycols Genapol® B and PN30 have very good lubricating properties, but also have the disadvantageous cloud point described. Examples 1-3 show that low molecular weight etherpyrrolidone carboxylic acids from the prior art that contain only PEG chains originating from ethylene oxide do not meet the requirements for an effective lubricant, since the wear marks at a use concentration of 10% in the case of these Reichert measurements ought to be at least less than 20 mm².

Examples 4-11 show the advantageous absence of a cloud point and comparable or superior lubricity effect of the etherpyrrolidone carboxylic acids of the invention. High molecular weight representatives (examples 6, 7, 9, 11) in particular exhibit improved lubricity effects. In one of the embodiments preferred as outlined, the etherpyrrolidone carboxylic acids have pure polypropylene chains (examples 4-6, 10, 11), and in high molecular weight form they have the best lubricity values, even at very low'concentrations (examples 6,11).

The following examples illustrate the production of a cooling lubricant composition concentrate comprising etherpyrrolidone carboxylic acids, and the use of said concentrate.

### Example 12

### Cooling Lubricant Composition Concentrate (Comparative)

A 250 ml beaker is charged with 40 g of water. With continual stirring, first 15 g of triethanolamine (neutralizing agent), then 20 g of isononanoylamidocaproic acid (corrosion inhibitor), 9 g of Genapol B, and 1 g of Genapol PN30 are added. For clarification, 5 g of butyl diglycol are added.

### Example 13

### Cooling Lubricant Composition Concentrate Comprising etherpyrrolidone carboxylic acid from Example 6

A 250 ml beaker is charged with 45 g of water. With continual stirring, first 17 g of triethanolamine, then 20 g of isononanoylcaproic acid and then 8 g of the ECA from example 6 are added.

### Example 14

### Cooling Lubricant Composition Concentrate Comprising etherpyrrolidone carboxylic acid from Example 6

A 250 ml beaker is charged with 45 g of water. With continual stirring, first 15 g of triethanolamine and 2 g of monoethanolamine are added and then 12 g of isononanoylcaproic acid, 8 g of dithiodibenzoic acid (AW/EP additive), and lastly 8 g of the ECA from example 7 are added.

### Example 15

### Cooling Lubricant Composition Concentrate, Comprising etherpyrrolidone carboxylic acid from Example 11

A 250 ml beaker is charged with 45 g of water. With continual stirring, first 17 g of triethanolamine, then 20 g of isononanoylcaproic acid and then 8 g of the ECA from example 11 are added.

Table 2 sets out the performance data for the synthetic cooling lubricant compositions produced from the concentrates of the invention which comprise the most effective etherpyrrolidone carboxylic acids from example 6 and 11, and also for the prior art. The pH levels were adjusted to 8.5 with traces of monoethanolamine.

TABLE 2
Cooling lubricant compositions from concentrates

| | Example 12 | Example | Example | Example |
|---|---|---|---|---|
| Lubricant | (C) | 13 | 14 | 15 |
| pH | 8.5 | 8.5 | 8.5 | 8.5 |
| 5% in water | | | | |
| Hard water stability | | | | |
| 4% in water of 40° dH¹⁾ | clear | clear | clear | clear |
| 4% in water of 60° dH | clear | clear | clear | clear |
| 4% in water of 80° dH | clear | clear | clear | clear |
| Corrosion control | 4% | 4% | 4% | 4% |
| DIN 51360/2; conc. | | | | |
| for rating of 0 | | | | |
| Lubricity test²⁾ | 22.5 mm² | 20.5 mm² | 18.5 mm² | 20.2 mm² |
| Reichert balance | | | | |
| Cloud point | 41° C. | >95° C. | >95° C. | >95° C. |

¹⁾dH = German hardness
²⁾loading 1.5 kg, steel rollers, 100 m travel, rotary speed 1.6 m/s; blank value for water: 38 mm²

Table 2 shows the effectiveness of the cooling lubricant compositions produced from the cooling lubricant composition concentrates comprising the ether carboxylic acids (example 13-15), in comparison to the prior art (example 12). The ether carboxylic acids are used together with the neutralizing agent, corrosion inhibitors, and optionally EP/AW additive (example 14) in smaller quantities than the polyalkylene glycols (Genapol PN 30, Genapol B). Corrosion control and stability with respect to hard water are not impaired.

1. An etherpyrrolidone carboxylic acid of the formula (1) 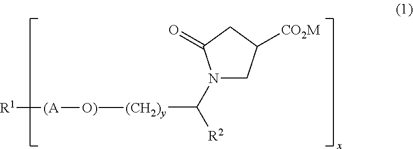
in which
R¹ R¹ is a radical obtained by the formal abstraction of the hydroxyl hydrogen atom from a di-, tri- or tetrahydric alcohol comprising 2 to 6 carbon atoms, or by the formal abstraction of the hydroxyl hydrogen atom from a monoalcohol selected from the group consisting of methanol, ethanol, n-propanol, n-butanol, isopropanol, isobutanol and tert-butanol
R² is H, CH₃, CH₂CH₃
(A-O) is a (poly)alkylene glycol unit containing one or more C₂-C₄ alkoxy groups or a mixture of plural C₂-C₄ alkoxy groups, at least one alkoxy group being a butoxy or propoxy group
M is hydrogen, alkali metal, alkaline earth metal or an ammonium group
y is 1 or 2
x is 1, 2, 3 or 4.

2. An etherpyrrolidone carboxylic acid as claimed in claim 1, in which R¹ is an aliphatic group.

3. An etherpyrrolidone carboxylic acid as claimed in claim 1, in which R¹ contains 2 to 6 carbon atoms.

4. An etherpyrrolidone carboxylic acid as claimed in claim 1, in which (A-O) is an alkoxy group which has a number-average molecular weight of more than 200 g/mol.

5. An etherpyrrolidone carboxylic acid as claimed in claim 1, in which (A-O) is an alkoxy group which comprises more than 20 mol % of propoxy groups.

6. An etherpyrrolidone carboxylic acid as claimed in claim 1, in which y is 1 and R² is H, CH₃ or CH₂CH₃.

7. An etherpyrrolidone carboxylic acid as claimed in claim 1, in which y is 2 and R² is H.

8. A composition comprising at least one etherpyrrolidone carboxylic acid as claimed in claim 1, water and a neutralizing agent in an amount sufficient for the pH of the composition to be between 7 and 11.

9. A composition as claimed in claim 8, further comprising 0.1%-60% by weight of a water-soluble corrosion inhibitor.

10. A composition as claimed in claim 8, further comprising 0.1% and 20% by weight of an EP/AW additive.

11. A cooling lubricant comprising an etherpyrrolidone carboxylic acid as claimed in claim 1 as a lubricant.
